(19) United States
(12) Patent Application Publication
(10) Pub. No.: US 2015/0299739 A1
(43) Pub. Date: Oct. 22, 2015

(54) PROCESSES FOR PRODUCING FERMENTATION PRODUCTS

(71) Applicant: SHELL OIL COMPANY, Houston, TX (US)

(72) Inventors: Peter John Edward HARLICK, Houston, TX (US); David Matthew BROWN, Amsterdam (NL)

(21) Appl. No.: 14/686,815

(22) Filed: Apr. 15, 2015

Related U.S. Application Data
(60) Provisional application No. 61/980,857, filed on Apr. 17, 2014.

(51) Int. Cl. C12P 7/14

(52) U.S. Cl. CPC C12P 7/14; C12P 2203/00

(57) ABSTRACT

A method to produce a fermentation product comprising: providing a biomass material; pretreating the biomass material by contacting the biomass material with a solution containing at least one alpha-hydroxysulfonic acid to produce a pretreated biomass mixture containing at least one fermentable sugar; adding one or more saccharification enzymes to the pretreated biomass mixture, wherein the pretreated biomass mixture has a suitable condition for the one or more saccharification enzymes; performing enzymatic hydrolysis of cellulose by the one or more saccharification enzymes for greater than 24 hours to generate a hydrolysate; and adding at least one fermentation microorganism to the hydrolysate under a suitable condition for simultaneous saccharification and fermentation.

### CROSS-REFERENCE TO RELATED APPLICATION

The present non-provisional application claims the benefit of pending U.S. Provisional Patent Application Ser. No. 61/980,857, filed Apr. 17, 2014, the disclosures of which are incorporated by reference herein in their entirety.

### TECHNICAL FIELD

Embodiments provided by the present disclosure generally relate to production of fermentation product; particularly from a lignocellulosic material.

### BACKGROUND

This section is intended to introduce various aspects of the art, which may be associated with exemplary embodiments of the present invention. This discussion is believed to assist in providing a framework to facilitate a better understanding of particular aspects of the present invention. Accordingly, it should be understood that this section should be read in this light, and not necessarily as admissions of any prior art.

Lignocellulosic biomass offers a promising alternative to petroleum, providing renewable and “carbon neutral” sources of fuels, such as bioethanol, and of other traditionally petroleum-based products such as plastics. Lignocellulosic biomass can be enzymatically hydrolysed, in a process typically known as saccharification, to provide sugars for fermentation by certain fermentation microorganisms. Because of its complex chemical structure, lignocellulose usually requires some pre-treatment that renders cellulose fibers accessible to enzyme catalysis, such as cellulase enzymes. Pretreatment usually involves mechanical size reduction (for example by milling or grinding) and chemical treatment such as with an alkali or more particularly an acid. The pretreatment process typically introduces inhibitors that reduce the efficiency of the subsequent enzymatic hydrolysis process.

Enzymatic hydrolysis heavily influences overall costs of production of fermentation products from lignocellulosic biomass. As such, the ability to increase such production, either through increasing the efficiency of saccharifying enzymes or improving overall production yields is desired. There have been many approaches to improve enzymatic efficiency. For instance, there have been attempts to reduce the hindering effects of lignin in lignocellulose material, typically including either by modifying the properties of lignin or by removing it altogether. Processes for removing lignin, however, have tended to be complex and costly. For example adding a solvent washing substrate after pretreatment to remove soluble and residual lignins increases both cost and complexity to the process.

Other attempts include the addition of surfactants, either during the pretreatment process or more usually at the hydrolysis stage. For example, WO-A-2009/095781 discloses the use of a PEG or surfactant as an additive during the hydrolysis of pretreated lignocellulosic biomass. It suggests that the effect of the additive can be particularly great at high (>20%) dry matter concentrations. WO-A-2008/134037 describes the addition of surfactants, at elevated temperatures and both with and without acid, in order to enhance the enzymatic digestibility of pretreated plant material. Surfactants, however, can cause foaming which may negatively impact saccharification and fermentation, and can also be expensive at its effective dosage. Further additional attempts include recycling enzymes, such as that disclosed in WO 2011125056, which adds to the complexity of an operation, as well as costs.

These approaches, however, often fail to prolong the activity of the saccharifying enzymes due to various inhibitors in a separate hydrolysis process with subsequent fermentation, which is also known as separate hydrolysis and fermentation (SHF). Generally, the hydrolysis yields depend on the type and pretreatment of the substrate, type and dosage of the enzyme and the hydrolysis time. An alternative to SHF is to combine the hydrolysis and fermentation to one single step, resulting in the process concept termed simultaneous saccharification and fermentation (SSF), which can address certain inhibitive effects of SHF. However, SSF suffers from certain disadvantages, including not providing the optimal conditions for hydrolysis and fermentation since it is a compromise between the two processes. Even when saccharifying enzymes are added before a fermentation microorganism is added in a SSF process to achieve incomplete or partial saccharification. For instance, US2011/0318803 discloses a hybrid saccharification and fermentation (HSF) step where saccharifying enzymes are added one to four hours before a fermentation microorganism was added. Similarly, U.S. Pat. No. 7,754,456 discloses a process where a first saccharifying enzyme preparation is added for a pre-hydrolysis step and a second saccharifying enzyme preparation is added for SSF. In addition, U.S. Pat. No. 7,754,456 requires separation of fibrous material from the pretreated biomass for saccharification, thereby introduce additional steps, and associated complications, to the ethanol production operation, as well as requiring additional amounts of enzymes which can drive up the operation costs.

Accordingly, it would be desirable to provide an economical method for improving the overall fermentation product yield generated from lignocellulosic material.

### SUMMARY

According to one aspect, there is provided a method to produce a fermentation product comprising: providing a biomass material; pretreating the biomass material by contacting the biomass material with a solution containing at least one alpha-hydroxysulfonic acid to produce a pretreated biomass mixture containing at least one fermentable sugar; adding one or more saccharification enzymes to the pretreated biomass mixture, wherein the pretreated biomass mixture has a suitable condition for the one or more saccharification enzymes; performing enzymatic hydrolysis of cellulose by the one or more saccharification enzymes for greater than 24 hours to generate a hydrolysate; and adding at least one fermentation microorganism to the hydrolysate under a suitable condition for simultaneous saccharification and fermentation to generate a fermentation product.

In some embodiments, the method further comprises removing at least a portion of the alpha-hydroxysulfonic acid from the pretreated biomass material by heating and/or reducing pressure to produce an acid-removed product containing at least one fermentable sugar substantially free of the a-hydroxysulfonic acid. In one embodiment, the removing comprises reverting the alpha-hydroxysulfonic acid to its components. In one embodiment, the method further comprises recycling the components for use in pretreating the biomass material.

In some embodiments, the alpha-hydroxysulfonic acid is present in an amount of from about 1% wt. to about 55% wt., based on the solution. In some embodiments, the alpha-hydroxysulfonic acid is produced from (a) a carbonyl compound or a precursor to a carbonyl compound with (b) sulfur dioxide or a precursor to sulfur dioxide and (c) water. In some embodiments, the pretreating step is carried out at a temperature within the range of about 50° C. to about 150° C. and a pressure within the range of 1 barg to about 10 barg. In one embodiment, the biomass is contacted with the alpha-hydroxysulfonic acid at a temperature of 120° C. or less. In some embodiments, the alpha-hydroxysulfonic acid is in-situ generated.

According to another aspect, there is provided another method to produce a fermentation product comprising: providing a pretreated biomass mixture comprising at most 0.50 wt % of a component selected from a group consisting of hydroxyl-methyl-furfural, furfural, and a combination thereof; adding one or more saccharification enzymes to the pretreated biomass mixture, wherein the pretreated biomass mixture has a suitable condition for the one or more saccharification enzymes; performing enzymatic hydrolysis of cellulose by the one or more saccharification enzymes for greater than 24 hours to generate a hydrolysate; and adding at least one fermentation microorganism to the hydrolysate under a suitable condition for simultaneous saccharification and fermentation to produce a fermentation product.

In some embodiments, the enzymatic hydrolysis is performed for greater than 48 hours. In some embodiments, the enzymatic hydrolysis is performed for greater than 72 hours. In some embodiments, the enzymatic hydrolysis is performed for at least 96 hours. In some embodiments, the hydrolysis is performed for at least 144 hours. In some embodiments, the pretreated biomass mixture is not subject to a washing step prior to the addition of one or more saccharification enzymes.

In some embodiments, the hydrolysate is not subject to a solids removal step prior to the addition of the at least one fermentation microorganism. In some embodiments, no additional saccharification enzymes are added after the addition of the at least one fermentation microorganism.

In some embodiments, the enzymatic hydrolysis is carried out for a period of time sufficient to achieve at least 75% net conversion of cellulose to glucose. In some embodiments, at least a portion of the remaining up to 25% of cellulose is hydrolyzed during simultaneous saccharification and fermentation.

In some embodiments, the pretreated biomass mixture comprises an amount of undissolved solids (UDS) in a range of about 5 wt % to 30 wt %. In one embodiment, the method further comprises separating at least a solid fraction and an aqueous fraction from the pretreated biomass mixture, wherein the solid fraction comprises a high solids/liquid mixture containing at least 12 wt % undissolved solids based on the solid fraction, and the aqueous fraction comprises a bulk liquid stream containing fermentable sugar; and mixing the solid fraction and aqueous fraction together to achieve said amount of undissolved solids in a range of about 5 wt % to 30 wt %.

Other features of embodiments of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

These drawings illustrate certain aspects of some of the embodiments of the invention, and should not be used to limit or define the invention.

FIG. 1 schematically illustrates a block flow diagram of one exemplar embodiment to produce fermentation products.

FIG. 2 is a graph showing xylose consumption, glucose consumption, by-product formation, and ethanol production between active hydrolysis enzyme and inactive hydrolysis enzyme in fermentation.

### DETAILED DESCRIPTION

Embodiments of the present invention improve yield of production of fermentation products from a biomass feedstock by allowing for both separate enzymatic hydrolysis and subsequent simultaneous saccharification and fermentation (SSF). In one embodiment, a method for ethanol production comprises: (a) pretreating a biomass with a solution containing at least one alpha-hydroxysulfonic acid to generate a pretreated biomass comprising a plurality of polysaccharides; (b) contacting the pretreated biomass with at least one saccharification enzyme, which catalyzes enzymatic hydrolysis of the polysaccharides to generate a hydrolysate or a hydrolyzed product comprising one or more fermentable sugars; and (c) providing at least one fermentation microorganism to the hydrolyzed product in an environment configured for simultaneous hydrolysis and fermentation (SSF). In one embodiment, during SSF, at least one saccharification enzyme continues to catalyze polysaccharides in the hydrolyzed product to generate fermentable sugars to be metabolized by the fermentation microorganism.

The main advantage of a separate hydrolysis and separate fermentation stages is that the hydrolysis can be carried out at the optimum conditions, e.g., temperature, for the enzymes, and the fermentation can be carried out at the optimum conditions for the fermentation microorganism. A disadvantage of SHF is that compounds in the hydrolysis reaction, remnants from the pretreatment or generated during hydrolysis, can inhibit enzymatic activity during hydrolysis itself after a certain period of time. This inhibition of enzymes translates to a requirement of greater amounts of enzyme to achieve a hydrolysis yields target since allowing hydrolysis to continue to reach that target is often not an option if the enzymes have been inhibited.

In the simultaneous saccharification and fermentation process (SSF), saccharification and fermentation takes place at the same time in the same reactor. The main advantages offered by SSF include enhanced rate of cellulose hydrolysis due to uptake of sugars by the fermentation microorganism, which can reduce inhibition of the saccharification enzyme. The disadvantages of SSF, as mentioned above, include compromised conditions that are not optimal for either hydrolysis or fermentation because the optimal conditions for the saccharification enzyme and fermentation microorganism do not typically overlap.

Embodiments provided in this disclosure takes advantage of the benefits of both SHF and SSF by allowing for a separate hydrolysis reaction (SHF) to increase the yield of fermentable sugars under conditions optimal for hydrolysis and for the saccharification process to continue into fermentation, where there is a positive cycle of production of sugars fueling ethanol production and the uptake of sugars decreasing inhibitors for continued saccharification.

As used herein, the articles “a”, “an”, and “the” are intended to be nonrestrictive regarding the number of instances (i.e., occurrences) of the element or component. Therefore “a”, “an” and “the” should be read to include one or at least one, and the singular word form of the element or component also includes the plural unless the number is obviously meant to be singular.

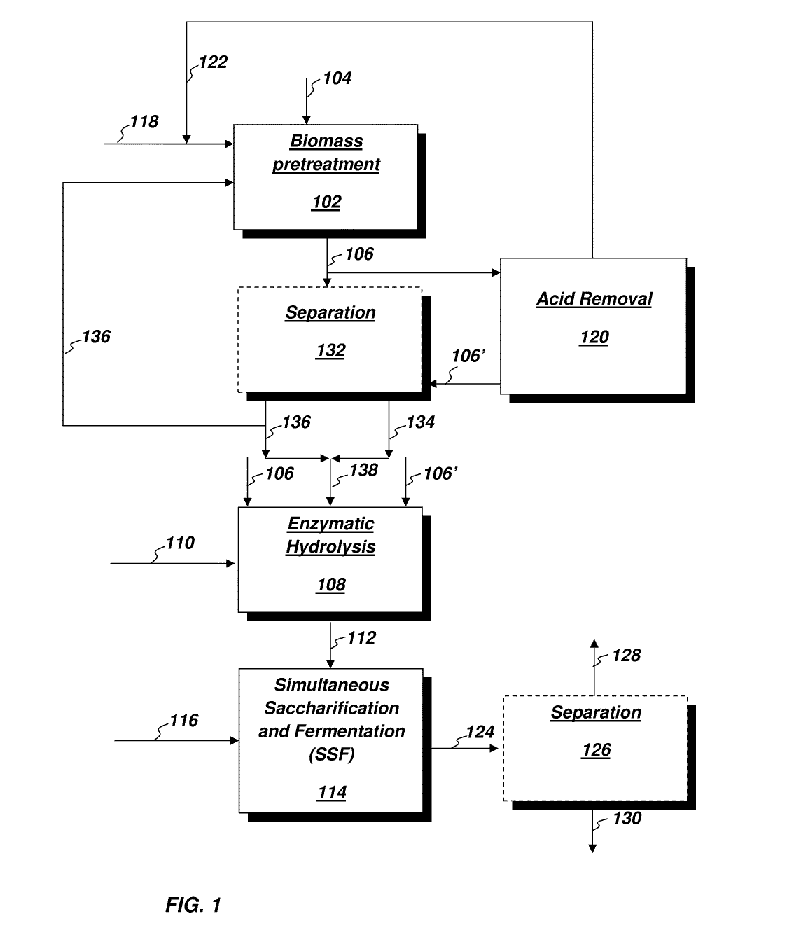

FIG. 1 provides an illustration of one exemplary embodiment, process 100, which comprises biomass pretreatment 102, enzymatic hydrolysis 108, and SSF 114. For biomass pretreatment 102, biomass 104 is contacted with pretreatment medium 118 containing α-hydroxysulfonic acid, which is effective in rendering biomass 104 susceptible to enzymatic saccharification as well as hydrolyzing at least a portion of biomass 104 to fermentable sugars like pentose, such as xylose. Biomass pretreatment 102 generates pretreated biomass mixture 106 containing biomass 104 and pretreatment medium 118 with the α-hydroxysulfonic acid. At least a portion of pretreated biomass mixture 106 is routed to enzymatic hydrolysis 108 where pretreated biomass mixture 106 is provided with at least one saccharification enzymatic mixture 110 to generate hydrolysate 112. In addition or alternatively, at least a portion of pretreated biomass mixture 106 is routed to acid removal 120 where at least a portion of the α-hydroxysulfonic acid is removed from pretreated biomass mixture 106. In acid removal 120, the α-hydroxysulfonic acid is reverted back to its components 122, which can be recycled in component form or in the recombined acid form for use in biomass pretreatment 102. While FIG. 1 shows components 122 with pretreatment medium 118, it is understood that, in addition to or alternatively, at least a portion of components 122 can enter biomass pretreatment 102 separate from pretreatment medium 118.

As shown, pretreated biomass mixture 106 exits acid removal 120 as pretreated biomass mixture 106′, preferably substantially without the α-hydroxysulfonic acid, to enter enzymatic hydrolysis 108 where pretreated biomass mixture 106′ and/or pretreated biomass mixture 106 is provided with at least one saccharification enzymatic mixture 110 to generate hydrolysate 112. After enzymatic hydrolysis 108, hydrolysate 112 is routed to SSF 114 where at least one fermentation microorganism 116 is provided to hydrolysate 112 under conditions configured to allow simultaneous saccharification and fermentation to generate fermentation product 124. In one embodiment, pretreated biomass mixture 106′ and/or pretreated biomass mixture 106 is not subject to a washing step (e.g., with water) before entering enzymatic hydrolysis 108 that other conventional methods typically employ to remove contaminants that can inhibit enzymatic hydrolysis, thereby allowing for a more efficient and cost effective process. In another embodiment, hydrolysate 112 is not subject to a separation step to remove solids in the hydrolysate prior to SSF 114. Hydrolysate 112 may be routed to SSF 114 directly from enzymatic hydrolysis 108. In such an embodiment, fermentation product 124 may be directed to separation zone 126 where volatile fermentation products 128, such as ethanol and other volatile organic compounds (VOC's) of interest, may be separated from the solids 130 in fermentation product 124.

Biomass

“Biomass” or “lignocellulosic biomass” refers to any cellulosic or lignocellulosic material and includes materials comprising cellulose, and optionally further comprising hemicellulose, lignin, starch, oligosaccharides and/or monosaccharides.

Biomass may also comprise additional components, such as protein and/or lipid. Biomass may be derived from a single source, or biomass can comprise a mixture derived from more than one source; for example, biomass could comprise a mixture of corn cobs and corn stover, or a mixture of grass and leaves. Biomass includes, but is not limited to, bioenergy crops (e.g., poplars, willows, switch grass, alfalfa, prairie bluestream, corn, soybean, algae and seaweed), agricultural residues (e.g., corn stalks, straw, seed hulls, sugarcane leavings, bagasse, nutshells, and manure from cattle, poultry, and hogs), municipal solid waste (e.g., waste paper), industrial solid waste, sludge from paper manufacture, yard waste, wood and forestry waste (e.g., wood or bark, sawdust, timber slash, and mill scrap). Examples of biomass include, but are not limited to, corn cobs, crop residues such as corn husks, corn stover, grasses, wheat, wheat straw, barley straw, hay, rice straw, switchgrass, waste paper, sugar cane bagasse, sorghum, components obtained from milling of grains, trees, branches, roots, leaves, wood chips, sawdust, shrubs and bushes, vegetables, fruits, flowers and animal manure.

In one embodiment, biomass comprises cellulose in an amount greater than about 20%, more preferably greater than about 30%, more preferably greater than about 40% (w/w). For example, biomass may comprise from about 20% to about 50% (w/w) cellulose, or any amount in between. In another embodiment, biomass comprises lignin in an amount greater than about 10%, more typically in an amount greater than about 15% (w/w).

Biomass Pretreatment

Biomass pretreatment 102 disrupts the fiber structure of biomass 104 and increases the surface area of biomass 104 to make it accessible to saccharification enzymatic mixture 110 in enzymatic hydrolysis 108. In a preferred embodiment, pretreatment 102 is performed so that a high degree of hydrolysis of the hemicellulose and a small amount of conversion of cellulose to glucose occur, thereby allowing the cellulose to be hydrolyzed to glucose in enzymatic hydrolysis 108 by saccharification enzymatic mixture 110. In one embodiment, the amount of cellulose hydrolysis is from 3 to 15 wt %. In another embodiment, the extent of xylan hydrolysis in biomass pretreatment 102 may be between about 80-100 wt %, that is at least 80 wt % and up to 100 wt %, or any range in between.

Referring to FIG. 1, in biomass pretreatment 102, at least a portion of biomass 104 is contacted with pretreatment medium 118 containing at least one α-hydroxysulfonic acid. In a preferred embodiment, the at least one α-hydroxysulfonic acid is effective for rendering biomass 104 susceptible to enzymatic hydrolysis at lower temperature, e.g., about 100 degrees C., as compared to conventional acid pretreatment processes while producing little to no furfural. Further, the α-hydroxysulfonic acid is reversible to readily removable and recyclable materials unlike mineral acids such as sulfuric, phosphoric, or hydrochloric acid. The lower temperatures and pressures employed in the biomass treatment leads to lower equipment cost.

The lower temperature process (for example, about 100° C. for α-hydroxymethane sulfonic acid or α-hydroxyethane sulfonic acid) reduces the rate of C₅ and C₆ sugar decomposition to other species such as furfural. Thus, free sugars can be introduced (via recycle) into the front end of a low temperature process and they will pass largely unchanged through pretreatment. This allows build up of high concentrations of steady state sugars while handling lower consistency in the pretreatment process using higher concentrations of α-hydroxysulfonic acids, enabling the recycle and build up of sugars in pretreatment step 102 for certain embodiments. Further, the ability to recycle fragile pentose sugars from the end of pretreatment to the inlet of pretreatment, without their subsequent conversion to undesirable materials such as furfural, allows lower consistencies in the pretreatment reaction itself, yet still passing a high consistency solids mixture containing high soluble sugars out of pretreatment.

The alpha-hydroxysulfonic acids of the general formula

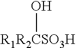

where R₁ and R₂ are individually hydrogen or hydrocarbyl with up to about 9 carbon atoms that may or may not contain oxygen can be used in the treatment of the instant invention. The alpha-hydroxysulfonic acid can be a mixture of the aforementioned acids. The acid can generally be prepared by reacting at least one carbonyl compound or precursor of carbonyl compound (e.g., trioxane and paraformaldehyde) with sulfur dioxide or precursor of sulfur dioxide (e.g., sulfur and oxidant, or sulfur trioxide and reducing agent) and water according to the following general equation 1.

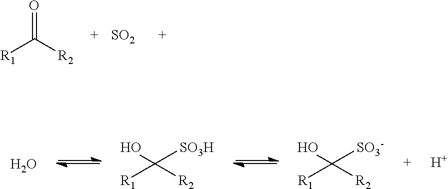

where R₁ and R₂ are individually hydrogen or hydrocarbyl with up to about 9 carbon atoms or a mixture thereof.

Illustrative examples of carbonyl compounds useful to prepare the alpha-hydroxysulfonic acids used in this invention are found where

R₁═R₂═H (formaldehyde)

R₁═H, R₂═CH₃ (acetaldehyde)

R₁═H, R₂═CH₂CH₃ (propionaldehyde)

R₁═H, R₂═CH₂CH₂CH₃ (n-butyraldehyde)R₁═H, R₂═CH(CH₃)₂ (i-butyraldehyde)

R₁═H, R₂═CH₂OH (glycolaldehyde)

R₁═H, R₂═CHOHCH₂OH (glyceraldehdye)

R₁═H, R₂═C(═O)H (glyoxal)

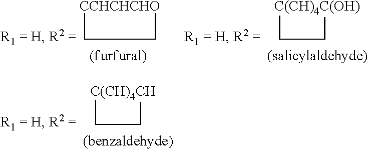

R₁═R₂═CH₃ (acetone)

R₁═CH₂OH, R₂═CH₃ (acetol)

R₁═CH₃, R₂═CH₂CH₃ (methyl ethyl ketone)

R₁═CH₃, R₂═CHC(CH₃)₂ (mesityl oxide)

R₁═CH₃, R₂═CH₂CH(CH₃)₂ (methyl i-butyl ketone)

R₁, R₂═(CH₂)₅ (cyclohexanone) or

R₁═CH₃, R₂═CH₂C₁ (chloroacetone)

The carbonyl compounds and its precursors can be a mixture of compounds described above. For example, the mixture can be a carbonyl compound or a precursor such as, for example, trioxane which is known to thermally revert to formaldehyde at elevated temperatures, metaldehdye which is known to thermally revert to acetaldehyde at elevated temperatures, or an alcohol that maybe converted to the aldehyde by dehydrogenation of the alcohol to an aldehyde by any known methods. An example of such a conversion to aldehyde from alcohol is described below. An example of a source of carbonyl compounds maybe a mixture of hydroxyacetaldehyde and other aldehydes and ketones produced from fast pyrolysis oil such as described in “Fast Pyrolysis and Bio-oil Upgrading, Biomass-to-Diesel Workshop”, Pacific Northwest National Laboratory, Richland, Wash., Sep. 5-6, 2006. The carbonyl compounds and its precursors can also be a mixture of ketones and/or aldehydes with or without alcohols that may be converted to ketones and/or aldehydes, preferably in the range of 1 to 7 carbon atoms.

The preparation of α-hydroxysulfonic acids by the combination of an organic carbonyl compounds, SO₂ and water is a general reaction and is illustrated in equation 2 for acetone.

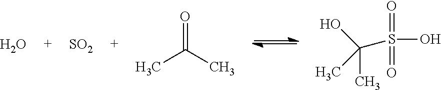

The α-hydroxysulfonic acids appear to be as strong as, if not stronger than, HCl since an aqueous solution of the adduct has been reported to react with NaCl freeing the weaker acid, HCl (see U.S. Pat. No. 3,549,319). The reaction in equation 1 is a true equilibrium, which results in facile reversibility of the acid. That is, when heated, the equilibrium shifts towards the starting carbonyl, sulfur dioxide, and water (component form). If the volatile components (e.g. sulfur dioxide) are allowed to depart the reaction mixture via vaporization or other methods, the acid reaction completely reverses and the solution becomes effectively neutral. Thus, by increasing the temperature and/or lowering the pressure, the sulfur dioxide can be driven off and the reaction completely reverses due to Le Chatelier's principle, the fate of the carbonyl compound is dependent upon the nature of the material employed. If the carbonyl is also volatile (e.g. acetaldehyde), this material is also easily removed in the vapor phase. Carbonyl compounds such as benzaldehyde, which are sparingly soluble in water, can form a second organic phase and be separated by mechanical means. Thus, the carbonyl can be removed by conventional means, e.g., continued application of heat and/or vacuum, steam and nitrogen stripping, solvent washing, centrifugation, etc. Therefore, the formation of these acids is reversible in that as the temperature is raised, the sulfur dioxide and/or aldehyde and/or ketone can be flashed from the mixture and condensed or absorbed elsewhere in order to be recycled. In embodiments provided by this disclosure, few undesired byproducts, such as furfurals, are produced as compared to pretreatment processes using other conventional mineral acids. In one embodiment, the temperature employed in biomass treatment allows for diminished formation of byproducts such as furfural or hydroxymethylfurfural. In another embodiment, substantial removal of the α-hydroxysulfonic acid from the pretreated biomass mixture following treatment minimizes complications to downstream processing caused by the neutralization of the acid with a base and formation of salts.

It has been found that the position of the equilibrium given in equation 1 at any given temperature and pressure is highly influenced by the nature of the carbonyl compound employed, steric and electronic effects having a strong influence on the thermal stability of the acid. More steric bulk around the carbonyl tending to favor a lower thermal stability of the acid form. Thus, one can tune the strength of the acid and the temperature of facile decomposition by the selection of the appropriate carbonyl compound.

In one embodiment, the acetaldehyde starting material to produce the alpha-hydroxysulfonic acids can be provided by converting ethanol, produced from the fermentation of the treated biomass of the invention process, to acetaldehyde by dehydrogenation or oxidation. Dehydrogenation may be typically carried out in the presence of copper catalysts activated with zinc, cobalt, or chromium. At reaction temperatures of about 260-290° C., the ethanol conversion per pass is 30-50% and the selectivity to acetaldehyde is between 90 and 95 mol %. By-products include crotonaldehyde, ethyl acetate, and higher alcohols. Acetaldehyde and unconverted ethanol are separated from the exhaust hydrogen-rich gas by washing with ethanol and water. Pure acetaldehyde is recovered by distillation, and an additional column is used to separate ethanol for recycle from higher-boiling products. It may not be necessary to supply pure aldehyde to the α-hydroxysulfonic acid process above and the crude stream may suffice. The hydrogen-rich off-gas is suitable for hydrogenation reactions or can be used as fuel to supply some of the endothermic heat of the ethanol dehydrogenation reaction. The copper-based catalyst has a life of several years but requires periodic regeneration. In an oxidation process, ethanol maybe converted to acetaldehyde in the presence of air or oxygen and using a silver catalyst in the form of wire gauze or bulk crystals. Typically, the reaction is carried out at temperatures in a range from about 500 to 600 degrees C., depending on the ratio of ethanol to air. Part of the acetaldehyde is also formed by dehydrogenation, with further combustion of the hydrogen to produce water. At a given reaction temperature, the endothermic heat of dehydrogenation partly offsets the exothermic heat of oxidation. Ethanol conversion per pass is typically between 50 and 70%, and the selectivity to acetaldehyde is in the range of 95 to 97 mol %. By-products include acetic acid, CO and CO₂. The separation steps are similar to those in the dehydrogenation process, except that steam is generated by heat recovery of the reactor effluent stream. The off-gas steam consists of nitrogen containing some methane, hydrogen, carbon monoxide and carbon dioxide; it can be used as lean fuel with low calorific value. An alternative method to produce acetaldehyde by air oxidation of ethanol in the presence of a Fe—Mo catalyst. The reaction can be carried out at a temperature in a range of about 180 to 240 degrees C. and atmospheric pressure using a multitubular reactor. In one embodiment, selectivities to acetaldehyde between 95 and 99 mol % can be obtained with ethanol conversion levels above 80%.

Various factors can affect the conversion of biomass 104 in pretreatment 102. The carbonyl compound or incipient carbonyl compound (such as trioxane) with sulfur dioxide and water should be added in an amount and under conditions effective to form alpha-hydroxysulfonic acids. The temperature and pressure of the hydrolysis reaction should be in the range to form alpha-hydroxysulfonic acids and to hydrolyze lignocellulosic biomass into fermentable sugars. The amount of carbonyl compound or its precursor and sulfur dioxide should be to produce alpha-hydroxysulfonic acids in the range from about 1 wt %, preferably from about 5 wt % to about 55 wt %, preferably to about 40 wt %, more preferably to about 20 wt %, based on the total solution. For the reaction, excess sulfur dioxide is not necessary, but any excess sulfur dioxide may be used to drive the equilibrium in eq. 1 to favor the acid form at elevated temperatures. The contacting conditions of the hydrolysis reaction may be conducted at temperatures preferably at least from about 50° C. depending on the alpha-hydroxysulfonic acid used, although such temperature may be as low as room temperature depending on the acid and the pressure used. The contacting condition of the hydrolysis reaction may range preferably up to and including about 150° C. depending on the alpha-hydroxysulfonic acid used. In a more preferred condition the temperature is at least from about 80° C., most preferably at least about 100° C. In a more preferred condition the temperature range up to and including about 90° C. to about 120° C. The reaction is preferably conducted at as low a pressure as possible, given the requirement of containing the excess sulfur dioxide. The reaction may also be conducted at a pressure as low as about 1 barg, preferably about 4 barg, to about pressure of as high as up to 10 barg. The temperature and pressure to be optimally utilized will depend on the particular alpha-hydroxysulfonic acid chosen and optimized based on economic considerations of metallurgy and containment vessels as practiced by those skilled in the art.

Numerous methods have been utilized by those skilled in the art to circumvent these obstacles to mixing, transport and heat transfer. Thus weight percentage of lignocellulosic biomass solids to total liquids (consistency) in biomass 104 may be as low as 1% or higher depending on the apparatus chosen and the nature of the biomass (even as high as 33% if specialized equipment is developed or used). The solids percent is weight percent of dry solids basis and the wt % liquids contains the water in biomass 104. In a preferred embodiment, where a more conventional equipment is desired, then the consistency of biomass 104 may be from at least 1 wt %, preferably at least about 2 wt %, more preferably at least about 8 wt %, up to about 25 wt %, preferably to about 20 wt %, more preferably to about 15 wt %.

The temperature of pretreatment 102 can be chosen so that the maximum amount of extractable carbohydrates are hydrolyzed and extracted as fermentable sugar (more preferably pentose and/or hexose) from the lignocellulosic biomass feedstock while limiting the formation of degradation products. It is known in the art that the temperatures required for pretreatment 102 depend on the reaction time, the pH of the solution (acid concentration), and the reaction temperature. Thus, if the acid concentration is raised, the temperature may be reduced and/or the reaction time extended to accomplish the same objective. The advantages of lowering the reaction temperature are that the fragile monomeric sugars are protected from degradation to dehydrated species such as furfurals and that the lignin sheath is not altered and re-deposited upon the lignocellulosic biomass. If high enough levels of acid are employed, temperatures can be reduced below the point at which sugar degredation or lignin deposition are problematic; this in turn is made possible through the use of reversible α-hydroxysulfonic acids.

In some embodiments, pretreatment 102 can employ a plurality of reactor vessels (not shown). These vessels may have any design capable of carrying out a hydrolysis reaction. Suitable reactor vessel designs can include, but are not limited to, batch, trickle bed, co-current, counter-current, stirred tank, down flow, or fluidized bed reactors. Staging of reactors can be employed to arrive the most economical solution. In another embodiment, a series of reactor vessels may be used with an increasing temperature profile so that a desired sugar fraction is extracted in each vessel. The outlet of each vessel can then be cooled prior to combining the streams, or the streams can be individually fed to the next reaction for conversion.

Suitable reactor designs can include, but are not limited to, a backmixed reactor (e.g., a stirred tank, a bubble column, and/or a jet mixed reactor) may be employed if the viscosity and characteristics of the partially digested bio-based feedstock and liquid reaction media is sufficient to operate in a regime where bio-based feedstock solids are suspended in an excess liquid phase (as opposed to a stacked pile digester). It is also conceivable that a trickle bed reactor could be employed with biomass 104 present as the stationary phase and pretreatment medium 118 passing over biomass 104.

In some embodiments, pretreatment 102 is carried out in any system of suitable design, including systems comprising continuous-flow (such as CSTR and plug flow reactors), batch, semi-batch or multi-system vessels and reactors and packed-bed flow-through reactors. For reasons strictly of economic viability, it is preferable that pretreatment step 102 uses a continuous-flow system at steady-state equilibrium. In one advantage of the process in contrast with the dilute acids pretreatment reactions where residual acid is left in the reaction mixture (<1% wt. sulfuric acid), the lower temperatures employed using these acids (5 to 20% wt.) results in substantially lower pressures in the reactor resulting in potentially less expensive processing systems such as plastic lined reactors, duplex stainless reactors, for example, such as 2205 type reactors.

The preferable residence time of the lignocellulosic biomass to contact with the α-hydroxysulfonic acid in the hydrolysis reaction system may be in the range of about 5 minutes to about 4 hours, most preferably about 15 minutes to about 1 hour. Pretreated biomass mixture 106 from pretreatment 102 contains fermentable sugar or monosaccharides, such as pentose and/or hexose that is suitable for further processing. The term “fermentable sugar(s)” refers to oligosaccharides and monosaccharides that can be used as a carbon source by a microorganism in a fermentation process.

Referring to FIG. 1, acid removal step 120 removals at least a portion of the α-hydroxysulfonic acid from pretreated biomass mixture 106 by reverting at least a portion of the α-hydroxysulfonic acid in pretreated biomass mixture 106 to its components 122, which can be recycled in component form and/or in its recombined form for use in biomass pretreatment 102.

In acid removal step 120, the residual α-hydroxysulphonic acid can be removed from pretreated biomass mixture 106 by application of heat and/or vacuum to reverse the formation of α-hydroxysulphonic acid to its starting material to pretreated biomass mixture 106′, which is substantially free of the α-hydroxysulfonic acid. In some embodiments, pretreated biomass mixture 106′ being substantially free of α-hydroxysulphonic acid means no more than about 2 wt % is present in pretreated biomass mixture 106′, preferably no more than about 1 wt %, more preferably no more than about 0.2 wt %, most preferably no more than about 0.1 wt % present in pretreated biomass mixture 106′. The temperature and pressure of acid removal step 120 will depend on the particular α-hydroxysulphonic acid used and minimization of temperatures employed are desirable to preserve the sugars obtain in treatment reactions. Typically removal step 120 may be conducted at temperatures in the range from about 50° C., preferably from about 80° C., more preferably from 90° C., to about 110° C., up to about 150° C. The pressure may be in the range of from about 0.5 bara, to about 2 barg, more preferably from 0.1 barg to about 1 barg. It can be appreciated by a person skill in the art that pretreatment step 102 and acid removal step 120 can occur in the same vessel or a different vessel or in a number of different types of vessels depending on the reactor configuration and staging as long as the system is designed so that the reaction is conducted under condition favorable for the formation and maintainence of the α-hydroxysulfonic acid and removal favorable for the reverse reaction (as components). As an example, the reaction in a reactor vessel of pretreatment step 102 can be operated at approximately 100° C. and a pressure of 4 barg in the presence of α-hydroxyethanesulfonic acid and a reactor vessel of acid removal step 120 can be operated at approximately 110° C. and a pressure of 0.5 barg. It is further contemplated that the reversion can be favored by the reactive distillation of the formed α-hydroxysulfonic acid. In the recycling of the removed acid, optionally additional carbonyl compounds, SO2, and water may be added as necessary. The removed starting material and/or α-hydroxysulphonic acid, represented as components 122, may be condensed and/or scrubbed by contact with water and recycled to pretreatment step 102 as components or in its recombined form.

Biomass pretreated mixture 106 and/or 106′ may be optionally routed to separation step 132 where a high solids/liquid mixture is separated from biomass pretreated mixture 106 and/or 106′ to form at least solids fraction 134 and aqueous fraction 136. Solid fraction 134 may be characterized as a wet solids stream and aqueous fraction 136 may be characterized as a bulk liquid stream, which may contain soluble components of pretreated biomass mixture 106 and/or 106′. Separation step 132 can be carried out by any suitable separation method to separate wet solids and liquids. Examples of suitable separation method, for example, may include centrifugal force, filtration, decantation, and other like methods. Another exemplary method to separate the soluble components of pretreated biomass mixture 106 and/or 106′ from the solids is washing pretreated biomass mixture 106 and/or 106′ with an aqueous solution to produce a wash stream comprising fermentable sugars released during pretreatment 102, which may be routed directly to SSF 114 (not shown). In a preferred embodiment, pretreated biomass mixture 106 and/or 106′ is not washed before it is subject to enzymatic hydrolysis 108.

In one embodiment, solids fraction 134 can contain at least 12 wt % undissolved solids containing cellulose, preferably in the range of 15 wt % to 35 wt % undissolved solids, and more preferably in the range of 20 wt % to 25 wt % undissolved solids, based on solids fraction 134. In another embodiment, aqueous fraction 136 may constitute up to 20 to 80% of the liquid from the acid-removed product stream that contains fermentable sugar (e.g., pentose and optionally hexose). In one embodiment, at least a portion of aqueous fraction 136 is recycled to pretreatment step 102 where aqueous fraction 136 comprises greater than about 2 wt %, preferably about 5 wt % or greater, more preferably about 8 wt % or greater, of sugar based on aqueous fraction 136. Solids fraction 134 may be recycled in such a manner as to keep biomass 104 pumpable, such as about 15 wt % or less of solids content, in pretreatment step 102. At least a portion of aqueous fraction 136 can be used to dilute biomass 104 in pretreatment step 102 at any point, e.g., near an inlet or outlet of a reactor. At least a portion of solids fraction 134 may optionally be provided to a wash system (not shown) that may have one or more washing steps with water. At least a portion of solids fraction 134 and aqueous fraction 136 may be combined with each other to produce pretreated biomass mixture 138 having the desired properties, such as the amount of undissolved solids.

In some embodiments, pretreatment step 102 and/or acid removal step 120 can be operated in a continuous or semi-continuous manner rather than batch processes. In certain embodiments, a wash step may not be necessary due to the composition of product stream and the wet solids stream having low levels of enzyme inhibitors or inactivators. If a wash step is employed, a liquid wash stream (not shown in figure) may be separated from the washed wet solids and be passed back to pretreatment step 102. In one embodiment, pretreated biomass mixture 106′ substantially without alpha-hydroxysulfonic acid is routed to separation step 132 and/or enzymatic hydrolysis step 108, along with pretreated biomass mixture 106 or by itself.

Pretreated biomass mixture 106 and/or 106′ may have low level of enzyme inhibitors or inactivators. For example, exemplary inhibitors or inactivators include, but not limited to, hydroxy-methyl-furfural and/or furfural, and properties of lignin exhibited when heated to higher than about 135 degrees C. As is known to one skilled in the art of cellulosic pretreatment, pretreatment temperatures higher than the glass transition of lignin (about 135 degrees C.) typically result in fiber characteristics that are toxic to current commercially available cellulases compositions due to the production of inhibitors or inactivators to enzymatic hydrolysis. The amount of hydroxyl-methyl-furfural (HMF) in pretreated biomass mixture 106 and/or 106′ may be in a range of 0 to 0.50 wt % based on pretreated biomass mixture 106 and/or 106′; for example, at most 0.50 wt %, at most 0.45 wt %, at most 0.40 wt %, at most 0.35 wt %, at most 0.30 wt %, at most 0.25 wt %, at most 0.20 wt %, at most 0.15 wt %, at most 0.10 wt %, or at most 0.05 wt %, based on pretreated biomass mixture 106 and/or 106′; other examples also include at least 0.05 wt %, at least 0.10 wt %, at least 0.15 wt %, at least 0.20 wt %, at least 0.25 wt %, at least 0.30 wt %, at least 0.35 wt %, at least 0.40 wt %, at least 0.45 wt %, or at least 0.50 wt %, based on pretreated biomass mixture 106 and/or 106′. The amount of furfural in pretreated biomass mixture 106 and/or 106′ may be in a range of 0 to 0.50 wt % based on pretreated biomass mixture 106 and/or 106′; for example, at most 0.50 wt %, at most 0.45 wt %, at most 0.40 wt %, at most 0.35 wt %, at most 0.30 wt %, at most 0.25 wt %, at most 0.20 wt %, at most 0.15 wt %, at most 0.10 wt %, or at most 0.05 wt %, based on pretreated biomass mixture 106 and/or 106′; other examples also include at least 0.05 wt %, at least 0.10 wt %, at least 0.15 wt %, at least 0.20 wt %, at least 0.25 wt %, at least 0.30 wt %, at least 0.35 wt %, at least 0.40 wt %, at least 0.45 wt %, or at least 0.50 wt %, based on pretreated biomass mixture 106 and/or 106′. One non-limiting exemplary way to determine the amount of HMF and/or furfural in a pretreated biomass mixture is to measure the amount of HMF in an aqueous fraction of that pretreated biomass mixture and calculate the amount of HMF in the pretreated biomass mixture using methods known to one of ordinary skill in the art. The low amount of contaminants may also allow hydrolysate 112 to be route to SSF 114, without a washing step, from enzymatic hydrolysis 108.

Enzymatic Hydrolysis

Referring to FIG. 1, enzymatic hydrolysis 108 provides pretreated biomass mixture 106′ and/or 106 with saccharification enzymatic mixture 110 to generate hydrolysate 112, which comprises fermentable sugars converted from cellulose and one or more enzymes from saccharification enzymatic mixture 110. In one embodiment, saccharification enzymatic mixture 110 comprises a cellulase enzyme. By the term “cellulase enzymes” or “cellulases,” it refers to a mixture of enzymes that hydrolyze cellulose. The mixture may include cellobiohydrolases (CBH), glucobiohydrolases (GBH), endoglucanases (EG), glycosyl hydrolyase family 61 proteins (GH61) and β-glucosidase. By the term “β-glucosidase”, it is meant any enzyme that hydrolyzes the glucose dimer, cellobiose, to glucose. In a non-limiting example, a cellulase mixture may include EG, CBH, GH61 and β-glucosidase enzymes

Saccharification enzymatic mixture 110 may also comprises one or more xylanase enzymes. Examples of xylanase enzymes that may also be used for this purpose and include, for examples, xylanase 1, 2 (Xyn1 and Xyn2) and β-xylosidase, which are typically present in cellulase mixtures.

Saccharification enzymatic mixture 110 may comprise any type of cellulase enzymes, regardless of their source. Non-limiting examples of cellulases which may be used include those obtained from fungi of the genera Aspergillus, Humicola, and Trichoderma, Myceliophthora, Chrysosporium and from bacteria of the genera Bacillus, Thermobifida and Thermotoga. In some embodiments, the filamentous fungal host cell is an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

It is well known in the art that microorganisms that produce polysaccharide-hydrolyzing enzymes often exhibit an activity, such as cellulose degradation, that is catalyzed by several enzymes or a group of enzymes (or an “enzyme consortium”) having different substrate specificities. Thus, a “cellulase” from a microorganism may comprise a group of enzymes, one or more or all of which may contribute to the cellulose-degrading activity. Commercial or non-commercial enzyme preparations, such as cellulase, may comprise numerous enzymes depending on the purification scheme utilized to obtain the enzyme preparation. One or more enzymes in saccharification enzymatic mixture 110 may be unpurified and provided as a type of cell extract or whole cell preparation. Saccharification enzymatic mixture 110 may be produced using recombinant microorganisms that have been engineered to express multiple saccharifying enzymes.

In one embodiment, enzymatic hydrolysis 108 is carried out in an environment that is optimal for saccharification enzymatic mixture 110. In one embodiment, enzymatic hydrolysis 108 is preferably carried out in a suitable aqueous environment under conditions which can readily be determined by one skilled in the art.

In a preferred embodiment, the pH of pretreated biomass mixture 106 and/or 106′ in enzymatic hydrolysis 108 is adjusted so that it is within a range which is optimal for saccharification enzymatic mixture 110. Generally, the pH of the pretreated feedstock is adjusted to within a range of about 3.0 to about 8.0, or any pH in between, such as pH of 4-7, including pH 5 and 6.

The temperature of pretreated biomass mixture 106 and/or 106′ in enzymatic hydrolysis 108 is adjusted so that it is within the optimum range for the activity of saccharification enzymatic mixture 110. Generally, a temperature of about 15° C. to about 100° C., about 20° C. to about 85° C., about 30° C. to about 70° C., preferably between 40 and 60 degrees C., especially around 50° C., or any temperature there between, is suitable for most saccharification enzymes. Saccharification enzymatic mixture 110, comprising enzymes such as cellulases, β-glucosidase and other accessory enzymes, can be added to pretreated biomass mixture 106 and/or 106′, prior to, during, or after the adjustment of the temperature and pH of pretreated biomass mixture 106 and/or 106′ after pretreatment 102. Preferably saccharification enzymatic mixture 110 is added to pretreated biomass mixture 106 and/or 106′ in enzymatic hydrolysis 108 after the temperature and pH adjustment.

In one embodiment, enzymatic hydrolysis 108 is carried out for greater than 24 hours, greater than 48 hours, greater than 72 hours, greater than 96 hours, greater than 120 hours, greater than 144 hours, greater than 168 hours, greater than 192 hours. In another embodiment, the hydrolysis time is between 72 and 300 hours, preferably 96 to 200 hours, more preferably between 96 and 144 hours. That is, in embodiments provided by this disclosure, one or more enzymes in saccharification enzymatic mixture 110 remains active and hydrolyzes cellulose to glucose for greater than 24 hours, greater than 48 hours, greater than 72 hours, greater than 96 hours, greater than 120 hours, greater than 144 hours, greater than 168 hours, greater than 192 hours. In another embodiment, one or more enzymes in saccharification enzymatic mixture 110 remains active and hydrolyzes cellulose to glucose from 72 to 300 hours, such as 96 to 200 hours, 96 to 144 hours, or 144 to 168 hours, 168 to 192 hours, or 192 to 300 hours. In one embodiment, enzymatic hydrolysis 108 is carried out for a period of time sufficient to achieve at least 75%, 80%, 85%, 90%, or 95% net conversion of total available cellulose in biomass feed 104 to glucose. That is, in some embodiments, pretreatment 102 can provide at least a portion of the at least 75% conversion while enzymatic hydrolysis 108 provides the remaining conversion to achieve the desired net conversion amount.

In embodiments provided herein, one or more enzymes in saccharification enzymatic mixture 110 can remain active and prolong hydrolysis of pretreated biomass mixture 106 and/or 106′ as compared to biomass pretreated with other conventional acids. Not intending to be tied to theory, it is believed this is because pretreated biomass mixture 106 and/or 106′ contains less inhibitors than biomass pretreated with other conventional acids, and/or higher pretreatment temperatures.

In certain embodiments where hydrolysis is carried out for greater than 96 hours, the amount of saccharification enzymatic mixture 110 used can be reduced to achieve a desired hydrolysis target yield as compared to a hydrolysis reaction carried out for a lesser amount of time. This is because hydrolysis yields generally depend on reaction time and enzyme amount for a particular pretreated biomass and type(s) of enzyme. That is, to achieve a target hydrolysis yield, more enzymes can be used to reach the target with less reaction time, or vice versa, less enzyme can be used if hydrolysis can be carried out longer to allow the enzyme time to continue the hydrolysis process, if the enzyme remains active for the duration of the hydrolysis.

One skilled in the art would know how to determine the effective amounts of enzymes to use for enzymatic hydrolysis 108, and adjust conditions for optimal enzyme activity in the SSF. One skilled in the art would also know how to optimize the classes of enzyme activities required to obtain optimal saccharification of a given pretreated biomass under the selected conditions. The cellulase enzyme dosage is chosen to convert the cellulose of the pretreated feedstock to glucose. For example, an appropriate cellulase dosage can be about 1 to about 100 mg enzyme (dry weight) per gram of cellulose, for example, about 0.1, 0.5, 1, 2, 3, 4, 5, 6, 7, 8, 9, 10, 15, 20, 25, 30, 35, 40, 45, 50, 55, 60, 65, 70, 75, 80, 85, 90, and 95 mg enzyme per gram of cellulose, or any value in between.

The pretreated biomass mixture entering enzymatic hydrolysis 108 (e.g., 106, 106′, and/or 138) may contain from about 5 wt % to about 30 wt % undissolved solids (UDS) or any range in between, based on the pretreated biomass mixture. For example, the pretreated biomass mixture entering enzymatic hydrolysis 108 (e.g., 106, 106′, and/or 138) may contain from about 5 to 30 wt % or from about 15 to 28 wt % UDS, or any range in between; for example, at least 5%, at least 10 wt %, at least 15 wt %, at least 20 wt %, at least 25 wt %, or at least 30 wt %; and for another example, at most 30 wt %, at most 25 wt %, at most 20 wt %, at most 15 wt %, at most 10 wt %, at most 5 wt %. The range may contain numerical limits of 5, 6, 7, 8, 9, 10, 11, 12, 13, 14, 15, 16, 17, 18, 19, 20, 21, 22, 23, 24, 25, 26, 27, 28, 29 or 30% (w/w) based on the pretreated biomass mixture. In one embodiment, the UDS can be measured using the methods provided by the National Renewable Energy Laboratory (NREL) in Technical Report NREL/TP-510-42627, published in March 2008 and entitled Determination of Insoluble Solids in Pretreated Biomass Material. The conditions of pretreatment step 102 may be adjusted to provide pretreated biomass mixture 106 and/or 106′ with the desired UDS. In addition to or alternatively, pretreated biomass mixture 106 and/or 106′ may be processed post-pretreatment to achieve the desired UDS. Non-limiting examples include adding or removing liquid from the pretreated biomass mixture to decrease or increase the UDS, respectively. The pretreated biomass mixture may also be separated into at least a solid fraction and an aqueous fraction as described above, and these fractions can be combined to produce the desired UDS.

Enzymatic hydrolysis step 108 may be carried out in a reactor system, which may include a series of hydrolysis reactors. The number of hydrolysis reactors in the system depends on the cost of the reactors, the volume of the hydrolysis reaction, and other factors. The hydrolysis may be carried out in multiple stages or may be performed in a single stage. One skilled in the art would know how to optimize the classes of enzyme activities required to obtain optimal saccharification of a given pretreated biomass under the selected conditions.

Simultaneous Saccharification and Fermentation (SSF)

Referring to FIG. 1, hydrolysate 112 is routed to simultaneous saccharification and fermentation (SSF) 114, where fermentation microorganism 116 is provided to hydrolysate 112 containing at least one saccharification enzyme from saccharification enzymatic mixture 110 of enzymatic hydrolysis 108. Additional media components such as sugars, salts, growth enhancers, and/or an antibiotic corresponding to an antibiotic resistance gene in fermentation microorganism 116 are not typically necessary but can be included.

In one embodiment, cellulose in hydrolysate 112 that has not been hydrolyzed during enzymatic hydrolysis 108 is hydrolyzed in SSF 114 as fermentation microorganism 116 metabolizes the fermentable sugars, generated from enzymatic hydrolysis 108 as well as generated during SSF 114, to produce fermentation product 124. In SSF 114, the enzymatic hydrolysis of cellulose in hydrolysate 112 and the fermentation of glucose to fermentation product 124, such as ethanol, are combined in one step (see, e.g., Philippidis, 1996, Cellulose bioconversion technology, Handbook on Bioethanol: Production and Utilization, Wyman, ed., Taylor & Francis, Washington, D.C., pp. 179-212).

That is, over the course of SSF 114, fermentable sugars already in hydrolysate 112 and those formed through enzymatic hydrolysis during SSF 114 are metabolized by fermentation microorganism 116 to produce fermentation product 124. In one embodiment, enzymatic hydrolysis 108 can be carried out until at least 75% of total available cellulose in biomass 104 has been converted to glucose, and the remaining up to 25% of cellulose is hydrolyzed during SSF 114. In one embodiment, metabolism of glucose by fermentation microorganism decreases the amount of glucose in the fermentation medium of SSF 114, which can allow for further continued hydrolysis as it is believed that glucose can act as an inhibitor to certain saccharification enzymes.

In an alternative embodiment, additional saccharification enzyme is not added during SSF 114. In that embodiment, the hydrolysis of the remaining cellulose in hydrolysate 112 is achieved by one or more enzymes of saccharification enzymatic mixture 110 added during enzymatic hydrolysis 108. Fermentation product 124 comprises any product generated by fermentation organism 116, including an alcohol, a sugar alcohol, an organic acid and a combination thereof.

Fermentation microorganism 116 can comprise any number of known microorganisms (for example, yeasts or bacteria) may be used to convert fermentable sugars to fermentation product 124. In an embodiment where fermentation product 124 comprises ethanol, fermentation microorganism 116 typically comprises a Saccharomyces species yeast. Glucose and any other hexoses present in the fermentation medium of SSF 114 may be fermented to ethanol by wild-type Saccharomyces cerevisiae, although genetically modified yeasts may be employed as well, as discussed below. The ethanol may then be distilled to obtain a concentrated ethanol solution. Fermentation product 124 comprising butanol may be produced from glucose by a microorganism such as Clostridium acetobutylicum and then concentrated by distillation. To further enhance ethanol yield, a yeast capable of converting C₅ sugars to ethanol can be introduced. Accordingly, in various embodiments, the yeast comprises a C6 sugar yeast and a C₅ sugar yeast. In some embodiments, the yeast comprises a yeast that is capable of converting C6 sugars and C₅ sugars to ethanol.

Xylose and arabinose that are derived from the hemicelluloses may also be fermented to ethanol by a yeast strain that naturally contains, or has been engineered to contain, the ability to ferment these sugars to ethanol. Examples of microbes that have been genetically modified to ferment xylose include recombinant Saccharomyces strains into which has been inserted either (a) the xylose reductase (XR) and xylitol dehydrogenase (XDH) genes from Pichia stipitis (U.S. Pat. Nos. 5,789,210, 5,866,382, 6,582,944 and 7,527,927 and European Patent No. 450530); or (b) fungal or bacterial xylose isomerase (XI) gene (U.S. Pat. Nos. 6,475,768 and 7,622,284). Examples of yeasts that have been genetically modified to ferment L-arabinose include, but are not limited to, recombinant Saccharomyces strains into which genes from either fungal (U.S. Pat. No. 7,527,951) or bacterial (WO 2008/041840) arabinose metabolic pathways have been inserted.

Fermentation product 124 can include organic acids such as lactic acid, citric acid, ascorbic acid, malic acid, succinic acid, pyruvic acid, hydroxypropanoic acid, itaconoic acid and acetic acid. In a non-limiting example, lactic acid is the fermentation product of interest. The most well-known industrial microorganisms for lactic acid production from glucose are species of the genera Lactobacillus, Bacillus and Rhizopus.

Moreover, xylose and other pentose sugars may be fermented to xylitol by fermentation microorganism comprising yeast strains selected from the group consisting of Saccharomyces, Kluyveromyces, Candida, Pichia, Schizosaccharomyces, Hansenula, Kloeckera, Schwanniomyces, Yarrowia, Aspergillus, Trichoderma, Humicola, Acremonium, Fusarium, and Penicillium. Bacteria are also known to produce xylitol, including Corynebacterium sp., Enterobacter liquefaciens and Mycobacterium smegmatis.

Moreover, exemplary fermentation microorganisms include Clostridia, Escherichia coli (E. coli) and recombinant strains of E. coli, genetically modified strain of Zymomonas mobilis such as described in US2003/0162271, U.S. Pat. Nos. 7,741,119 and 7,741,084 (which disclosures are herein incorporated by reference) are some examples of such bacteria. The microorganisms may further be a yeast or a filamentous fungus of a genus. The fermentation may also be performed with recombinant yeast engineered to ferment both hexose and pentose sugars to ethanol. Recombinant yeasts that can ferment one or both of the pentose sugars xylose and arabinose to ethanol are described in European Patent EP 1727890, European Patent EPI 863901 and WO 2006/096130 which disclosures are herein incorporated by reference. Xylose utilization can be mediated by the xylose reductase/xylitol dehydrogenase pathway (for example, WO9742307 A1 19971113 and WO9513362 A1 19950518) or the xylose isomerase pathway (for example, WO2007028811 or WO2009109631). It is also contemplated that the fermentation organism may also produce fatty alcohols, for example, as described in WO 2008/119082 and PCT/US07/011923 which disclosure is herein incorporated by reference. In another embodiment, the fermentation may be performed by yeast capable of fermenting predominantly C6 sugars for example by using commercially available strains such as Thermosacc and Superstart.

Preferably, SSF 114 is performed at or near the temperature and pH optima of the fermentation microorganism. For example, the temperature may be from about 25° to about 55° C., or any amount there between. The dose of fermentation microorganism 116 will depend on other factors, such as the activity of fermentation microorganism 116, the desired fermentation time, and other parameters. It will be appreciated that these parameters may be adjusted as desired by one of skill in the art to achieve optimal SSF conditions.

In certain embodiments, SSF 114 is performed at a temperature of between 25° C. and 50° C. For example, the SSF reaction takes place at a temperature of 25° C. or above, 28° C. or above, 30° C. or above, 32° C. or above, 35° C. or above, or 38° C. or above. For example, SSF 114 can take place at a temperature of 50° C. or below, 45° C. or below, 40° C. or below, 38° C. or below, 35° C. or below, or 30° C. or below. For example, SSF 114 can take place in a temperature range of from 28° C. to 45° C., such as from 30° C. to 40° C., from 32° C. to 38° C. In an exemplary embodiment, SSF 114 can be carried out at a temperature of from 32° C. to 35° C. In another embodiment, SSF 114 is carried out at a temperature of about 32° C. Moreover, the pH of the saccharification-fermentation mixture of SSF 114 may be adjusted and/or maintained from about 4 to about 6.5, from about 4 to about 6, from about 4 to about 5.5, from about 4.5 to about 6, from about 4.5 to about 5.5, or from about 5 to about 6. pH adjustment may occur batchwise or continuously by addition of acid or base to the vessel.

In general, SSF 114 may be operated on a continuous, batch, or fed-batch (e.g., including stepwise introduction of feed materials to the zone) basis. The configuration and type of the reactor(s) in SSF 114 may be readily selected by one skilled in the art. Preferably, reactors suitable for SSF 114 include those configured for continuous, batch or fed-batch operation (e.g., individual or a series of continuous stirred-tank reactors). In one embodiment, enzymatic hydrolysis 108 is carried out in a separate reactor as that of SSF 114. Alternatively, enzymatic hydrolysis 108 and SSF 114 are carried out in the same reactor.

In one embodiment employing batch fermentation processes, SSF 114 is conducted from start to finish in a single vessel. Alternatively, SSF processes can be carried out in continuous mode, which involves steady-state fermentation systems that operate without interruption, and wherein each stage of the SSF process occurs in a separate section of a given fermentation system, and flow rates are set to correspond to required residence times.

In certain embodiments, a fed-batch SSF process may be desirable. A fed-batch process entails a batch phase and a feeding phase. The culture medium of the batch phase and the culture medium added during the feeding phase are chemically defined, and the culture medium of the feeding phase is added, at least for a fraction of the feeding phase, at a feeding rate that follows a pre-defined exponential function, thereby maintaining the specific growth rate at a pre-defined value.

In one embodiment, SSF 114 can suitably proceed for a period of 3 to 7 days. For example, in a particular embodiment, SSF 114 can proceed from 1 day and up to 3, 4, 5, 6, or 7 days.

In certain aspects, the present disclosure provides a set of SSF conditions that are specifically suitable for use with a fermenting microorganism that is a fungus, for example, a S. cerevisiae. For example, these conditions include carrying out the reaction with a suitable yeast strain

Distillation

Distillation can be performed to recover fermentation product 124 such as, for example, ethanol. Any method known in the art can be used for recovery, including, but not limited to, chromatography {e.g., ion exchange, affinity, hydrophobic, chromatofocusing, and size exclusion), electrophoretic procedures {e.g., preparative isoelectric focusing), differential solubility {e.g., ammonium sulfate precipitation), SDS-PAGE, distillation, or extraction.

To facilitate a better understanding of the present invention, the following examples of preferred or representative embodiments are given. In no way should the following examples be read to limit, or to define, the scope of the invention.

### Example

In this Example, a process according to one embodiment provided by the present disclosure as follows:

Biomass feedstock corn stovers were obtained from Iron Horse Farms (Iowa, USA) as bales. These bales where then sized reduced with 1.5-inch screens. The size reduced corn stovers had a moisture content of about 10 wt %.

For the biomass pretreatment, the carbonyl compound or incipient carbonyl compound (such as trioxane) with sulfur dioxide and water were added in an amount and under conditions effective to form alpha-hydroxysulfonic acids. The amount of carbonyl compound or its precursor and sulfur dioxide were to produce alpha-hydroxysulfonic acids in the range about 5 wt % based on the total solution. The contacting conditions of the pretreatment with alpha-hydroxysulfonic acids were conducted at about 120° C. and a pressure of about 35 psig for about 60 minutes to generate pretreated biomass mixture, which had a slurry consistency. The pretreated biomass was then separated via filtration, without washing, into solids and aqueous fractions.

The resulting pretreated biomass solid and aqueous fractions were combined to obtain a 32% total solids mixture, which was about 20% UDS, and was directly treated, without any washing, with commercially available cellulases for 96 hours at about 50 to 55 degrees C. and pH of 4.8 to 5.5 to generate an enzymatic hydrolysate. The amount of cellulases added was about 10 mg of enzyme per gram of cellulose. The hydrolysate either entered simultaneously saccharification and fermentation directly or was heat treated at 80 degrees C. for about 1 hour to deactivate residual cellulase activity before fermentation to simulate enzymatic activities in enzymatic hydrolysate generated from other conventional pretreatment methods. Anaerobic fermentation with yeast was carried out at about 28 to 32 degrees C. at pH of about 5.0-6.0 for 4 days with the enzymatic hydrolysates, heat treated and non-heat treated. No solids removal step was performed prior to the addition of yeast according to suitable fermentation protocols known to one of ordinary skill in the art (e.g., using inoculation) to the enzymatic hydrolysates. Samples of the fermentation broth were taken at different times to measure the pH, and components profile, such as glucose, xylose, cellobiose, ethanol, acetic acid, and glycerol. The sampling times were at 0, 19, 43, and 76 hours.

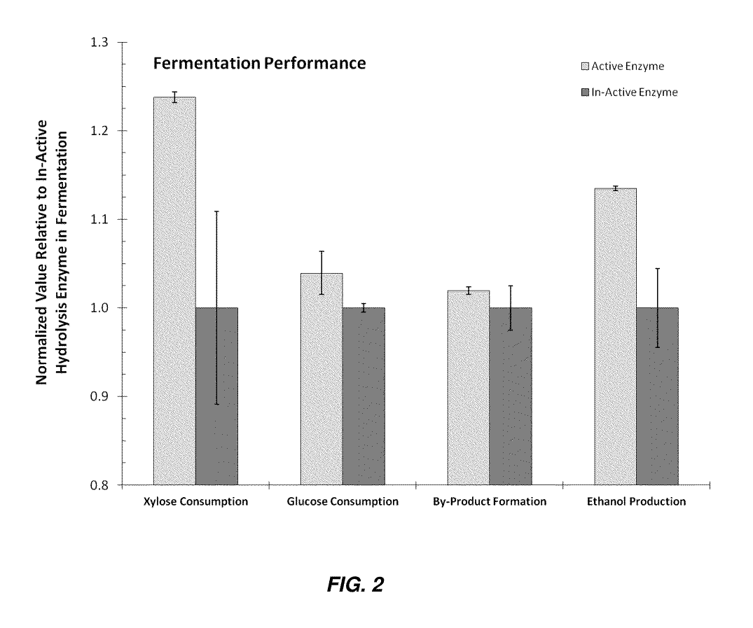

FIG. 2 shows the comparative values of xylose consumption, glucose consumption, by-product formation, and ethanol production during fermentation of the non-heat treated enzymatic hydrolysate (labeled as “active enzyme”) and of the heat treated enzymatic hydrolysate (labeled as “in-active enzyme”). As can be seen, simultaneous saccharification and fermentation took place in the fermentation broth with active enzymes, which were generated according to aspects of embodiments of the present invention, where additional xylose and glucose were being generated and consumed, which results in increased ethanol production. This is compared to the control—fermentation broth with inactive enzymes—which had less xylose and glucose consumption, and consequently, less ethanol production.

While embodiments of the invention are subject to modifications and alternative forms, specific embodiments thereof are shown by way of examples herein described in detail. It should be understood, that the detailed description thereto are not intended to limit the invention to the particular form disclosed, but on the contrary, the intention is to cover all modifications, equivalents and alternatives falling within the spirit and scope of the present invention as defined by the appended claims. The present invention will be illustrated by the following illustrative embodiment, which is provided for illustration only and is not to be construed as limiting the claimed invention in any way.

1. A method to produce a fermentation product comprising:
providing a biomass material;
pretreating the biomass material by contacting the biomass material with a solution containing at least one alpha-hydroxysulfonic acid to produce a pretreated biomass mixture containing at least one fermentable sugar;
adding one or more saccharification enzymes to the pretreated biomass mixture, wherein the pretreated biomass mixture has a suitable condition for the one or more saccharification enzymes;
performing enzymatic hydrolysis of cellulose by the one or more saccharification enzymes for greater than 24 hours to generate a hydrolysate; and
adding at least one fermentation microorganism to the hydrolysate under a suitable condition for simultaneous saccharification and fermentation to generate a fermentation product.

2. The method of claim 1 further comprising removing at least a portion of the alpha-hydroxysulfonic acid from the pretreated biomass material by heating and/or reducing pressure to produce an acid-removed product containing at least one fermentable sugar substantially free of the α-hydroxysulfonic acid.

3. The method of claim 2 wherein the removing comprises reverting the alpha-hydroxysulfonic acid to its components.

4. The method of claim 3 further comprising recycling the components for use in pretreating the biomass material.

5. The method of claim 1 wherein the alpha-hydroxysulfonic acid is present in an amount of from about 1% wt. to about 55% wt., based on the solution.
The method of claim 1 wherein the enzymatic hydrolysis is performed for greater than 48 hours.

6. The method of claim 1 wherein the enzymatic hydrolysis is performed for greater than 72 hours.

7. The method of claim 1 wherein the enzymatic hydrolysis is performed for at least 96 hours.

8. The method of claim 1 wherein the hydrolysis is performed for at least 144 hours.

9. The method of claim 1 wherein the pretreated biomass mixture is not subject to a washing step prior to the addition of one or more saccharification enzymes.

10. The method of claim 1 wherein the hydrolysate is not subject to a solids removal step prior to the addition of the at least one fermentation microorganism.

11. The method of claim 1 wherein no additional saccharification enzymes are added after the addition of the at least one fermentation microorganism.

12. The method of claim 1 wherein the enzymatic hydrolysis is carried out for a period of time sufficient to achieve at least 75% net conversion of cellulose to glucose.

13. The method of claim 12 wherein at least a portion of the remaining up to 25% of cellulose is hydrolyzed during simultaneous saccharification and fermentation.

14. The method of claim 1 wherein the pretreated biomass mixture comprises an amount of undissolved solids (UDS) in a range of about 5 wt % to 30 wt %.

15. The method of claim 14 further comprising:
separating at least a solid fraction and an aqueous fraction from the pretreated biomass mixture, wherein the solid fraction comprises a high solids/liquid mixture containing at least 12 wt % undissolved solids based on the solid fraction, and the aqueous fraction comprises a bulk liquid stream containing fermentable sugar; and
mixing the solid fraction and aqueous fraction together to achieve said amount of undissolved solids in a range of about 5 wt % to 30 wt %.

16. A method to produce a fermentation product comprising:
providing a pretreated biomass mixture comprising at most 0.50 wt % of a component selected from a group consisting of hydroxyl-methyl-furfural, furfural, and a combination thereof;
adding one or more saccharification enzymes to the pretreated biomass mixture, wherein the pretreated biomass mixture has a suitable condition for the one or more saccharification enzymes;
performing enzymatic hydrolysis of cellulose by the one or more saccharification enzymes for greater than 24 hours to generate a hydrolysate; and
adding at least one fermentation microorganism to the hydrolysate under a suitable condition for simultaneous saccharification and fermentation to produce a fermentation product.

17. The method of claim 16 wherein the enzymatic hydrolysis is performed for greater than 48 hours.

18. The method of claim 16 wherein the enzymatic hydrolysis is performed for greater than 72 hours.

19. The method of claim 16 wherein the enzymatic hydrolysis is performed for at least 96 hours.

20. The method of claim 16 wherein the hydrolysis is performed for at least 144 hours.

21. The method of claim 16 wherein the pretreated biomass mixture is not subject to a washing step prior to the addition of one or more saccharification enzymes.

22. The method of claim 16 wherein the hydrolysate is not subject to a solids removal step prior to the addition of the at least one fermentation microorganism.

23. The method of claim 16 wherein no additional saccharification enzymes are added after the addition of the at least one fermentation microorganism.

24. The method of claim 16 wherein the enzymatic hydrolysis is carried out for a period of time sufficient to achieve at least 75% net conversion of cellulose to glucose.

25. The method of claim 24 wherein at least a portion of the remaining up to 25% of cellulose is hydrolyzed during simultaneous saccharification and fermentation.

26. The method of claim 16 wherein the pretreated biomass mixture comprises an amount of undissolved solids (UDS) in a range of about 5 wt % to 30 wt %.

27. The method of claim 26 further comprising:
separating at least a solid fraction and an aqueous fraction from the pretreated biomass mixture, wherein the solid fraction comprises a high solids/liquid mixture containing at least 12 wt % undissolved solids based on the solid fraction, and the aqueous fraction comprises a bulk liquid stream containing fermentable sugar; and
mixing the solid fraction and aqueous fraction together to achieve said amount of undissolved solids in a range of about 5 wt % to 30 wt %.
